(19) United States
(12) Patent Application Publication
(10) Pub. No.: US 2026/0355069 A1
(43) Pub. Date: Nov. 12, 2026

(54) DENTAL COMPOSITIONS AND METHODS OF MAKING AND USING SAME

(71) Applicant: Solventum Intellectual Properties Company, Maplewood, MN (US)

(72) Inventors: Yizhong Wang, Woodbury, MN (US); Joel D. Oxman, Minneapolis, MN (US)

(21) Appl. No.: 19/103,594

(22) PCT Filed: Aug. 11, 2023
(86) PCT No.: PCT/IB2023/058145

§ 371 Date: Feb. 13, 2025

Related U.S. Application Data
(60) Provisional application No. 63/371,525, filed on Aug. 16, 2022.

(51) Int. Cl. A61K 6/889; A61K 6/76; A61K 6/77; A61K 6/836

(52) U.S. Cl. CPC A61K 6/889; A61K 6/76; A61K 6/77; A61K 6/836

(57) ABSTRACT

A multi-part hardenable dental composition includes a first part and a second part. The first part includes a mono- and/or multi-functional component having one or more ethylenically unsaturated group(s), acid-reactive glass particles; and an oxidizing agent. The second part includes a water miscible polyacid, a reducing agent that includes a water insoluble organic compound, and water.

### BACKGROUND

Two-part dental cements have been described in, for example, International Pub. WO2011/081975, International Pub. WO2020/75007A1, U.S. Pat. No. 5,501,727.

### DETAILED DESCRIPTION

Two-part glass ionomer cements have been in dental use for some time. Such materials are comprised of an ionic polymer component and a reactive glass component, which when mixed together in the presence of water undergo a cement setting reaction. These dental materials provide several desirable attributes including prolonged fluoride release, tolerance to moisture and saliva, good mechanical properties and excellent adhesion to dental hard tissues without pretreatments such as conditioners or adhesives. Powder-liquid, powder-paste, paste-paste, paste-liquid, and liquid-liquid two-part cements have been reported.

Various drawbacks have become evident with known materials and methods, including, for example, mechanical strength variability, varying consistencies, unsatisfactory working or setting times, cost per application, multiple dispensing and mixing steps, mechanical mixing equipment, and shelf life.

The materials described above are available as multi-part systems, typically in two-parts. These can be in any combination of powder, liquid, or paste. Shelf stability of the individual parts is extremely important so that there is no change in viscosity, color, or any other property occurring during the shelf life of the material (typically 2-4 years). In use, the parts are mixed together and then applied. Setting should occur in a short period of time so that the procedure is not uncomfortable for the patient or the operator. The setting characteristics should allow for sufficient time for mixing the materials and applying to the tooth preparation and/or prosthodontic or orthodontic device in place in the mouth.

Glass ionomer cement systems targeting stability have been developed as aqueous compositions and compositions having unique redox chemistry. Such compositions, however, have been associated with certain drawbacks. For example, aqeuous based pastes present challenges in containing water in the packaging and problems associated with the paste “drying out”. As another example, while tertiary amine and peroxide redox curing systems can provide effective curing of resin in glass ionomer cement systems, the amine/peroxide systems have been found to produce an undesirable color change in the composition. While compositions have been developed to mitigate color change issues via incorporation of encapsulated redox curing agents, such compositions involve more complexity in the chemistry and manufacturing. Consequently, there continues to be a growing interest in alternative methods and compositions for delivering glass ionomer cements and related materials in a more stable manner.

The present disclosure, at least in part, is directed to such alternative methods and compositions. Generally, the multi-part system of the present disclosure utilize a minimal (or no) amount of water in the first part to stabilize the water soluble metal persulfate salts in such part with no concern of water loss (i.e., the paste drying out). Additionally, a dispersion of 1-Benzyl-5-phenyl barbituric acid (BPBA) in an aqueous based second part that does not include a polymerizable monomer provides stability to the second part and preserves the reactivity of BPBA during storage. Still further, it was discovered that the BPBA based redox curing system produces no color change in the cured cement (in contrast to the above-discussed amine/peroxide based curing composition.

### DEFINITIONS

The term “water soluble” refers to a material, such as a monomer, which is partially or fully water soluble and dissolves in water alone in the amount of at least 5 g per liter of water at 25 °C.

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

Also herein, the recitations of numerical ranges by endpoints include all numbers subsumed within that range (e.g., the range of viscosity ratios 1:0.06 to 1:13 includes 1:0.06 to 1:13, 1:0.1 to 1:13, 1:0.25 to 1:13, 1:0.5 to 1:13, 1:0.6 to 1:13, 1:1 to 1:13, 1:0.06 to 1:10, 1:0.06 to 1:7.5, 1:0.06 to 1:5, 1:0.06 to 1:3.5, 1:0.06 to 1:1, 1:0.1 to 1:10, 1:0.25 to 1:7.5, 1:0.5 to 1:5, 1:0.6 to 1:3.5, 1:0.75 to 1:2, 1:0.9 to 1:1.1, etc.).

In some embodiments, the present disclosure is directed to a multi-part (e.g., two-part) hardenable dental composition (e.g., glass ionomer cement), the first part (sometimes referenced as paste A) including (i) liquid monofunctional and/or multifunctional monomers, oligomers, or polymers, (ii) an oxidizing agent; and (iii) acid-reactive glass, and the first part being free or substantially free of water; and the second part (sometimes referenced as paste B) including (i) a reducing agent; and (ii) a polyacid without a polymerizable sidechain group, (iii) water; and (iv) optionally, filler with no polymerizable groups attached to the surface.

Surprisingly, it was discovered that by separating the components of the ionic redox polymerization system (i.e., the reducing agent and the oxidizing agent) into different parts, a more reliable two-part cement system could be achieved, That is, it was discovered that by pairing the reducing agent and polyacid in a second part to form the dispersion of reducing agent in water to preserve the reactivity of the reducing agent, and by not including water (or at least not an appreciable amount of water) in the first part (that includes the water soluble oxidizing agent) a composition having all the benefits of known two-part cement compositions, but with enhanced stability (e.g., shelf life stability, even when subjected to handling errors (e.g., potential water loss due to loose fitting caps or failure to recouple cap to container in a timely fashion)) may be obtained.

In some embodiments, a first part of the multi-part hardenable composition may include (i) liquid mono- and/or multi-functional components having ethylenically unsaturated group(s) (sometimes referred to herein as a “resin system”), (ii) an oxidizing agent; and (iii) acid-reactive glass. In some embodiments, the first part may include no more than a small amount of water.

In some embodiments, the first part may be in the form of a paste. That is, not a powder or a liquid, but a mixture of liquid and non-dissolvable powder/solid components having a generally uniform composition.

In some embodiments, the first part may include either or both of liquid mono- and multi-functional (e.g., di-functional) components having ethylenically unsaturated group(s). The liquid mono- or multi-functional components may include monomers, oligomers, or polymers. In some embodiments, the liquid mono- or multi-functional components may be water soluble. The liquid mono- or multi-functional components may also be selected such that they are miscible with the other components of the hardenable composition. That is, these components may be at least sufficiently miscible that they do not undergo substantial sedimentation when combined with the other parts of the composition. In some embodiments, the first part may include both liquid mono- and multi-functional (e.g., di-functional) components having ethylenically unsaturated group(s).

In some embodiments, suitable ethylenically unsaturated groups include allyl, vinyl, acrylate, and methacrylate groups. In some embodiments, such monomers (or oligomers or polymers) have a relatively low molecular weight and include only one ethylenically unsaturated group per monomer molecule. In some embodiments, the molecular weight of such monomers is about 100 to about 1000. In some embodiments, including any one of the above embodiments which includes a water-soluble liquid mono-functional monomer having one ethylenically unsaturated group per monomer molecule, the water soluble liquid monomer may be selected from the group consisting of 2-hydroxyethyl (meth)acrylate, glycerol mono(meth)acrylate, sugar methacrylates, and a combination thereof.

Also as indicated above, the multi-part hardenable compositions described herein, in certain embodiments may also include a component having at least two ethylenically unsaturated groups per monomer molecule which provides some cross linking in the composition when hardened. In some embodiments, this monomer may have a viscosity less than bisphenol A-glycidyl methacrylate (Bis-GMA), or not more than 50 percent of Bis-GMA.

In some embodiments, suitable difunctional monomers may include glycerol dimethacrylate. Alternatively, or additionally, a water soluble monomer is used. Suitable water soluble dimethacrylates include various molecular weights of polyethylenglycol (dimeth)acrylates ranging from approximately 400 to 1000 weight average molecular weight.

The components of the resin system are selected such that they are miscible with the other components of the hardenable composition. That is, preferably, the components of the resin system are at least sufficiently miscible that they do not undergo substantial sedimentation when combined with the other ingredients of the composition (e.g., reducing agent and oxidizing agent). Preferably, the components of the resin system are miscible with water. The components of the resin system can be monomers, oligomers, polymers, or combinations thereof.

The components of the resin system are selected such that they are miscible with the other components of the hardenable composition. That is, preferably, the components of the resin system are at least sufficiently miscible that they do not undergo substantial sedimentation when combined with the other ingredients of the composition (e.g., reducing agent and oxidizing agent). Preferably, the components of the resin system are miscible with water. The components of the resin system can be monomers, oligomers, polymers, or combinations thereof.

In some embodiments, liquid mono- and multi-functional (e.g., di-functional) components having ethylenically unsaturated group(s) may be present in the first part in an amount of between 1 and 50 wt. %, between 3 and 40 wt. %, or between 5 and 30 wt. %, based on the total weight the first part.

In some embodiments, suitable oxidizing agents may include persulfates such as sodium, potassium, ammonium and alkyl ammonium persulfates, benzoyl peroxide, hydroperoxides such as cumene hydroperoxide, tert-butyl hydroperoxide, tert-amyl hydroperoxide and 2,5-dihydroperoxy-2,5-dimethylhexane, salts of cobalt (III) and iron (III), hydroxylamine, perboric acid and its salts, salts of a permanganate anion, and combinations thereof. Hydrogen peroxide can also be used, although it may, in some instances, interfere with the photoinitiator, if one is present. In some embodiments, the oxidizing agent may include potassium persulfate (e.g., milled potassium persulfate with small particle size for easy dispersion in the paste, such as an average particle size of less than 100 microns). The oxidizing agent may optionally be provided in an encapsulated form as described in U.S. Pat. No. 5,154,762. The oxidizing agent may be selected such that it is miscible in the compositions and/or miscible in water.

In some embodiments, the oxidizing agent may be present in the first part in an amount of between 0.01 and 10 wt. %, between 0.1 and 4.0 wt. %, or between 0.5 and 2.0 wt. %, based on the total weight the first part.

In some embodiments, the first part may include acid-reactive glass. Suitable acid-reactive glasses may include ion-leachable glasses, e.g., as described in U.S. Pat. Nos. 3,655,605; 3,814,717; 4,143,018; 4,209,434; 4,360,605 and 4,376,835. In some embodiments, the acid-reactive glass may be selected from borate glasses, phosphate glasses and fluoroaluminosilicate glasses. In some embodiments, the acid-reactive glass may include fluoroaluminosilicate (FAS) glass.

In embodiments that include FAS glass, the FAS glass may include sufficient elutable cations so that a hardened dental composition will form when the glass is mixed with the components of the hardenable composition. The glass may also include sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass may be in the form of particles that are sufficiently finely divided so that they can conveniently be mixed with the other cement components and will perform well when the resulting mixture is used in the mouth.

In some embodiments, the average particle size (average longest dimension-typically, diameter) for the FAS glass is no greater than 10 micrometers or no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT and KETAC-FIL (3M ESPE Dental Products, St. Paul, MN), FUJI II, GC FUJI LC and FUJI IX (G-C Dental Industrial Corp., Tokyo, Japan) and CHEMFIL Superior (Dentsply International, York, PA). Mixtures of fillers can be used if desired.

In some embodiments, the FAS glass can be subjected to a surface treatment. Suitable surface treatments include acid washing (e.g., treatment with a phosphoric acid), treatment with a phosphate, treatment with a chelating agent such as tartaric acid, and treatment with a silane or an acidic or basic silanol solution. Desirably the pH of the treating solution or the treated glass is adjusted to neutral or near-neutral, as this can increase storage stability of the hardenable composition.

In some embodiments, any of the above described acid-reactive glass particles may also be subjected to a surface treatment. Suitable surface treatments include acid washing, treatment with phosphates, treatment with chelating agents such as tartaric acid, treatment with a silane or silanol coupling agent. In some embodiments, the acid-reactive glass particles may be silanol treated fluoroaluminosilicate glass particles, as described in U.S. Pat. No. 5,332,429, the disclosure of which is incorporated by reference herein in its entirety.

In some embodiments, the acid-reactive glass may be present in the first part in an amount of between 1 and 80 wt. %, between 3 and 60 wt. %, or between 5 and 40 wt. %, or based on the total weight the first part.

In some embodiments, the first part may not include water or be substantially devoid of water. In this regard, in some embodiments, the first part may include water in an amount of less than 5 wt. %, less than 1 wt. %, or less than 0.5 wt. %, based on the total weight of the first part. In some embodiments, the first part may include no water other than that which is unintentionally introduced by the various components that comprise the first part (i.e., the first part may not include any added water).

In some embodiments, a second part of a multi-part hardenable composition may include (i) a polyacid (e.g., a polyacid without a polymerizable sidechain group); (ii) a reducing agent that includes 1-Benzyl-5-phenyl barbituric acid (BPBA); and (iii) water, and (iv) optionally filler with no polymerizable group attached to the filler surface. As with the first part, in some embodiments, the second part may be in the form of a paste.

In some embodiments, the second part may include a polyacid. In some embodiments, the polyacid may be water miscible. Suitable water miscible polyacids may include homo- or copolymers of unsaturated mono-, di-, and tricarboxylic acids, for example, homo- or copolymers of acrylic acid, itaconic acid or maleic acid. In some embodiments, the water miscible polyacids may include a polymer having sufficient pendent ionic groups to undergo a setting reaction in the presence of a reactive glass and water, and sufficient pendent non-ionically polymerizable groups to enable the resulting mixture to be cured by a redox curing mechanism and/or by exposure to radiant energy.

In some embodiments, the polyacid may be a (co) polymer (the reaction product of a polyacid with a coupling agent). In some embodiments, the polyacid may be the reaction product of a polymer selected from the group consisting of polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof.

In some embodiments, the polyacid may have Formula I:

B(X)ₘ(Y)n I
wherein B is an organic backbone, each X independently is an ionic group which can undergo a setting reaction in the presence of water and the acid-reactive glass particles, each Y independently is a non-ionically polymerizable group, m is at least 2, and n is at least 1. In some embodiments, X is —COOH and Y is a copolymer group that does not include an ethylenically unsaturated group. In some embodiments, the backbone B is an oligomeric or polymeric backbone of carbon-carbon bonds, optionally containing non-interfering substituents such as oxygen, nitrogen or sulfur heteroatoms. The term “non-interfering” refers to substituents or linking groups that do not unduly interfere with either the ionic or the non-ionic polymerization reaction. In some embodiments, B is a hydrocarbon backbone. X and Y groups can be linked to the backbone B directly or by means of any non-interfering linking group, such as substituted or unsubstituted alkylene, alkyleneoxyalkylene, arylene, aryleneoxyalkylene, alkyleneoxyarylene, arylenealkylene, or alkylenearylene groups. Alkylene and arylene refer to the divalent forms of alkyl and aryl, respectively. The linking group may also include linkages such as —OC(═O)—, —C(═O) NH—, —NH—C(═O)O—, —O—, and the like, and combinations thereof, wherein each of these may be used in either direction. In some embodiments, Y is attached to B via an amide linkage.

The polyacid of Formula I can be prepared according to a variety of synthetic routes, including, but not limited to, (1) reacting n X groups of a polymer of the formula B(X)ₘ₊ₙ with a suitable compound in order to form n pendent Y groups, (2) reacting a polymer of the formula B(X)ₘ at positions other than the X groups with a suitable compound in order to form n pendent Y groups, (3) reacting a polymer of the formula B(Y)ₘ₊ₙ or B(Y)ₙ, either through Y groups or at other positions, with a suitable compound in order to form m pendent X groups and (4) copolymerizing appropriate monomers, e.g., a monomer containing one pendent X groups and a monomer containing one pendent Y groups. The synthetic route (1) above is preferred. Such groups can be reacted by the use of a “coupling compound”, i.e., a compound containing both a Y group and a reactive group capable of reacting with the polymer through an X group, thereby covalently linking the Y group to the backbone B in a pendent fashion. Suitable coupling compounds are organic compounds, optionally containing non-interfering substituents and/or non-interfering linking groups between the Y group and the reactive group.

In some embodiments, polyacid may be present in the second part in an amount of between 2 and 50 wt. %, between 5 and 35 wt. %, or between 10 and 15 wt. %, based on the total weight the second part.

In some embodiments, the reducing agent may include a water insoluble organic reducing agent such as 1-Benzyl-5-phenyl barbituric acid (BPBA) or ascorbyl palmitate. Generally, it was discovered that such organic reducing agents can maintain their redox curing reactivity when dispersed in water to form a paste with other components in the second part. Water soluble reducing agents, in contrast, will lose their redox curing reactivity overtime when they are dissolved in water. The water insoluble organic reducing agents of the present disclosure may be dispersed as particles in water to achieve such storage stability. Ultimately, these water insoluble reducing agents may dissolve by interaction with the resin system (e.g., methacrylate resins) after mixing of the two parts. The redox pair from the first part and the second part may then produce free radicals to cure the resin system.

In some embodiments, the reducing agent may be present in the second part in an amount of between 0.1 and 8.0 wt. %, between 0.25 and 5.0 wt. %, or between 0.5, and 3.0 wt. %, based on the total weight the second part.

In some embodiments, the second part may include water. Water may be present in the second part in an amount of between 1 and 70 wt. %, between 2 and 60 wt. %, between 2 and 50 wt. %, between 5 and 40 wt. %, between 5 and 30 wt. %, or between 7 and 20 wt. %, based on the total weight the second part.

In some embodiments, nonreactive fillers may also be included in the compositions described herein to control viscosity as well as for other reasons, such as to achieve a desired appearance, impart desired strength properties, impart radiopacity, and the like. In some embodiments, including any one of the above embodiments, the first part, the second part, or both, may further include a non-reactive filler in an amount of between 5 and 60 wt. %, between 10 and 50 wt. %, or between 15 and 40 wt. %, based on the total weight the respective part.

In some embodiments, non-reactive fillers may be selected from one or more materials suitable for incorporation in compositions used for medical applications, such as fillers currently used in dental restorative compositions and the like. In some embodiments, the filler may have a maximum particle diameter less than 50 micrometers and an average particle diameter less than about 10 micrometers. The filler can have a unimodal or polymodal (e.g., bimodal) particle size distribution.

In some embodiments, the nonreactive filler is selected from the group consisting of inorganic material, crosslinked organic material, and a combination thereof. Suitable crosslinked organic materials are insoluble in the composition, and are optionally filled with inorganic filler. The filler should be non-toxic and suitable for use in the mouth. The filler can be radiopaque, radiolucent or non-radiopaque.

Examples of suitable non-reactive inorganic fillers are naturally-occurring or synthetic materials such as quartz, nitrides (e.g., silicon nitride), glasses derived from, for example, Ce, Sb, Sn, Zr, Sr, Ba and Al, colloidal silica, colloidal zirconia, feldspar, borosilicate glass, kaolin, talc, titania, and zinc glass; low Mohs hardness fillers such as those described in U.S. Pat. No. 4,695,251; and submicron silica particles (e.g., pyrogenic silicas such as the “Aerosil” Series “OX 50”, “130”, “150” and “200” silicas sold by Degussa and “Cab-O-Sil M5” silica sold by Cabot Corp.); metallic powders such as those disclosed in U.S. Pat. No. 5,084,491, especially those disclosed at column 2, lines 52-65; and combinations thereof.

Examples of suitable non-reactive organic filler particles include filled or unfilled pulverized polycarbonates, polyepoxides, and the like. Preferred non-reactive filler particles are quartz, submicron silica and zirconia, and non-vitreous microparticles of the type described in U.S. Pat. No. 4,503,169. Mixtures of these non-reactive fillers are also contemplated, as well as combination fillers made from organic and inorganic materials.

In some embodiments which include a nonreactive filler, the nonreactive filler is selected from the group consisting of fumed silica, zirconia-silica, quartz, nonpyrogenic silica, and combinations thereof.

In some embodiments, the surface of the non-reactive filler particles may be treated with a coupling agent in order to enhance the bond between the filler and polymerizable components when the composition is hardened. The use of suitable coupling agents include gamma-methacryloxypropyltrimethoysilane, gamma-mercaptopropyltriethoxysilane, gamma-aminopropyltrimethoxysilane, SILQUEST A-1230 (Momentive Performance Chemicals), and the like.

Additional components, which are suitable for use in the oral environment, may optionally be used in (either or both parts of) the multi-part hardenable compositions described herein. In one example, such components include solvents, cosolvents (e.g., alcohols) or diluents. In another example, indicators, dyes, pigments, inhibitors, accelerators, viscosity modifiers, wetting agents, tartaric acid, chelating agents, surfactants, buffering agents, stabilizers (including free-radical stabilizers), submicron silica particles, additives that impart fluorescence and/or opalescence, modifying agents that prolonged working time, and other materials that will be apparent to those skilled in the art may be used. Additionally, medicaments or other therapeutic substances can be optionally added to the compositions. Examples include whitening agents, breath fresheners, flavorants, fragrances, anticaries agents (e.g., xylitol), fluoride sources, remineralizing agents (e.g., calcium phosphate compounds), enzymes, anesthetics, clotting agents, acid neutralizers, chemotherapeutic agents, immune response modifiers, thixotropes, polyols, anti-inflammatory agents, antimicrobial agents, antifungal agents, agents for treating xerostomia, desensitizers, and the like of the type which may be used in dental compositions. Combinations of any of the above additives may also be used in the compositions described herein. The selection and amount of any one such additive can be determined by one of skill in the art according to the desired result.

Modifying agents which may prolong the time between the beginning of the setting reaction in a restoration and the time sufficient hardening has occurred to allow subsequent clinical procedures to be performed on the surface of the restoration include, e.g., alkanolamines such as ethanolamine and triethanolamine, and mono-, di-, and tri-sodium hydrogenphosphates. Modifying agents can be added to either part A or part B. When used, they are present at a concentration between about 0.1 to 10 percent by weight, based on the total composition weight.

Certain stabilizers provide color stability. Such stabilizers include oxalic acid, sodium metabisulfite, sodium bisulfite, sodium thoisulfate, metaphosphoric acid, and combinations thereof.

Free radical stabilizers can be used with a photoinitiator to prevent premature polymerization or to adjust the working time in free radically initiated compositions. Suitable examples of free radical stabilizers include, e.g., butylated hydroxytoluene (BHT) and methyl ethyl hydroquinone (MEHQ).

Viscosity modifiers include thickening agents. Suitable thickening agents include hydroxypropyl cellulose, hydroxymethyl cellulose, carboxymethylcellulose and its various salts such as sodium, and combinations thereof.

In some embodiments, either or both to the first and second parts may include a photoinitiator. In some embodiments, photoinitiator may be present not as a primary curing enabler but, rather, to allow a practitioner to expeditiously cure excess portions of the hardenable composition during use (e.g., an excess amount of the material that is present once a dental crown has been seated on a prepared tooth).

Generally, the photoinitiators may act as a source of free radicals when activated by heat or light. Such initiators can be used alone or in combination with one or more accelerators and/or sensitizers. Suitable photoinitiators (i.e., photoinitiator systems that include one or more compounds) include binary and ternary photoinitiators. In one example, a ternary photoinitiator may include an iodonium salt, a photosensitizer, and an electron donor compound as described in U.S. Pat. No. 5,545,676 (Palazzotto et al.). Examples of iodonium salts include diaryl iodonium salts, e.g., diphenyliodonium chloride, diphenyliodonium hexafluorophosphate, diphenyliodonium tetrafluoroborate, and tolylcumyliodonium tetrakis(pentafluorophenyl) borate. Examples of photosensitizers include monoketones and diketones that absorb some light within a range of about 400 nanometers to 520 nanometers, preferably 450 to 500 nanometers. Preferred are alpha diketones that absorb light within these ranges. Examples of such photosensitizers include camphoroquinone, benzil, furil, 3,3,6,6-tetramethylcyclohexanedione, phenanthraquinone, 1-phenyl-1,2-propanedione, and other 1-aryl-1-alkyl-1,2-ethanediones, and cyclic alpha diketones. Most preferred is camphoroquinone. Preferred electron donor compounds include substituted amines, e.g., ethyl dimethylaminobenzoate.

The photoinitiator, when utilized, should be present in an amount sufficient to provide the desired rate of polymerization. This amount will be dependent in part on the light source, the thickness of the layer to be exposed to radiant energy, and the extinction coefficient of the photoinitiator.

In some embodiments, each part of the multi-part hardenable dental composition described herein may have a viscosity which is balanced with respect to the other parts of the composition. In some embodiments, including any one of the above embodiments, the first part and second part may each, independently, have a viscosity not less than 6 pascal-second (Pas) and not greater than 100 Pas. In some embodiments, the ratio of second part to first part viscosity is 1:0.06 to 1:13. 1:0.6 to 1:3.5, or 1:0.9 to 1:1.6. For purposes of the present disclosure, viscosity is as measured on a TA instrument (AR G2), at room temperature, at a shear rate of 20/s, with a simple shear method.

### Method of Making a Hardened Dental Material

The multi-part hardenable compositions of the present disclosure may be formed by combining the first part and the second part. Any conventional mixing or combination techniques may be employed. For example, the first and second parts can be dispensed onto a mix pad and mixed by hand, can be mixed using an automix tip, or can be mixed by a rotary instrument

In some embodiments, the methods, devices, and compositions described herein are well suited for a number of dental applications, such as, for example, a luting cement used to anchor or hold a prosthetic device (e.g., crown, bridge, inlay, onlay, post, abutment, veneer, prosthetic tooth, and the like) in place in the mouth; a restorative or filler material used, for example, for filling a cavity; a thin film used, for example, as a liner on dentin and enamel or a sealant or sealing material on enamel; an orthodontic bracket adhesive; a band cement; and the like. In some embodiments, the multi-part hardenable dental composition is selected from the group consisting of a liner material, a luting material, a restorative material, an endodontic material, and a sealing material. For certain embodiments, including any one of the above embodiments, the multi-part hardenable dental composition is an orthodontic bracket adhesive material or band cement.

In some embodiments, the present disclosure provides methods of making and using the multi-part hardenable compositions described herein. For example, the hardenable compositions of the present disclosure (after mixing) can be used in methods of adhering or cementing (either intraorally or extraorally) a dental article (e.g., crown, bridge, orthodontic appliance) to a tooth or bone, as well as in methods of filling a tooth.

Objects and advantages of this disclosure are further illustrated by the following examples, but the particular materials and amounts thereof recited in these examples, as well as other conditions and details, should not be construed to unduly limit this disclosure.

### EXAMPLES

TABLE 1
Materials List

| ABBREVIATION/NAME | DESCRIPTION | SOURCE |
|---|---|---|
| BPBA | 1-benzyl 5-phenyl barbituric acid; CAS# 72846-00-5 | Alfa Aesar |
| BHT | butylated hydroxytoluene (>99%, FCC, FG); CAS# | Sigma-Aldrich |
| | 128-37-0 | |
| TCP | tricalcium phosphate, Ca₃(PO₄)₂; CAS# 7758-87-4 | Sigma-Aldrich |
| potassium persulfate | potassium persulfate, (K₂S₂O₈); CAS 7727-21-1 | Sigma-Aldrich |
| | (milled with ceramic milling media for 15 minutes, | |
| | then hand screened through 60 micron mesh to get the | |
| | milled PP powder) | |
| CPQ | Camphorquinone: CAS# 10373-78-1 | Sigma-Aldrich |
| DMAPE | 2-[4-(Dimethylamino)phenyl]ethanol, CAS# 50438- | Sigma-Aldrich |
| | 75-0 | |
| GDMA | glycerol dimethacrylate | Sigma-Aldrich |
| HEMA | 2-hydroxyethyl methacrylate | Sigma-Aldrich |
| R812S fumed silica | AEROSIL R812S, hydrophobic fumed silica | Evonik Industries |
| Silica A300 | ABROSIL 300 is a hydrophilic fumed silica with | Evonik Industries |
| | specific surface area of 300 m²/g; high purity | |
| | amorphous anhydrous colloidal silicon dioxide, USP; | |
| | CAS# 112945-52-5 | |
| Silica filler | 6-micron Silica filler | MRC-UNITEC Co. |
| (6-micron) | | Ltd. Japan |
| S/T coarse FAS filler | Silane treated fluoroaluminosilicate (FAS) reactive | Silane treated as |
| | glass filler; having a particle size of about 3 | described in U.S. Pat. |
| | micrometers. | No. 7,173,074, column |
| | | 13 (Mo-Sci Corp., |
| | | Columbia, MO) |
| S/T fine FAS filler | Silane treated fluoroaluminosilicate (FAS) reactive | Silane treated as |
| | glass filler; having a particle size of about 1 | described in U.S. Pat. |
| | micrometers. | No. 7,173,074, column |
| | | 13 (Mo-Sci Corp., |
| | | Columbia, MO) |
| YbF3 filler | Yitterbium fluoride-nano-sized powder; CAS# | Sukgyung AT Co. Ltd., |
| | 13760-80-0, gentle brush push through 74 micron | Korea |
| | nylon screen to get screened YbF3 particles. | |
| AA-MA copolymer | copolymer (1:1) of acrylic acid and maleic acid; | 3M Internal Preparation |
| | CAS# 26677-99-6 | |

Resin-Modified Glass Ionomer (RMGI) cements are two-part systems that when the two parts are combined, they react and harden (cure). The RMGI examples below were prepared as paste-paste type of RMGI, (Paste “A” and Paste “B”) two-part reactive systems.

### “Paste A” Preparations for Resin-Modified Glass Ionomer (RMGI) Dental Compositions

For paste A preparation, HEMA/GDMA/BHT/CPQ were first combined and dissolved together, then the other ingredients were added for a total amount of 100 grams of paste A, which was mixed with a speed mixer at 3000 rpm for 2 minutes and repeated (2 cycles) to form a uniform paste.

TABLE 2
Paste “A” Portion For A RMGI Cement Two-Part System

| | RMGI Paste “A” | | | | | |
|---|---|---|---|---|---|---|
| | EXA1 | EXA2 | EXA3 | EXA4 | EXA5 | EXA6 |
| | Paste | Paste A2 | Paste | Paste | Paste | Paste |
| | A1 | (CONTROL) | A3 | A4 | A5 | A6 |
| Components | % | % | % | % | % | % |
| GDMA | 8.10 | 10.00 | 9.00 | 7.00 | 31.80 | 7.00 |
| HEMA | 20.26 | 16.00 | 15.00 | 19.00 | 0 * | 14.00 |
| BHT | 0.03 | 0.05 | 0.05 | 0.05 | 0.05 | 0.10 |
| CPQ | 0.10 | 0.12 | 0.12 | 0.10 | 0.15 | 0.10 |
| amine monomer 3-10- | 0.00 | 0.00 | 0.00 | 0.00 | 0.00 | 4.00 |
| 2021-PIL-1 | | | | | | |
| TCP | 0.00 | 0.00 | 0.00 | 0.25 | 0.00 | 0.00 |
| potassium persulfate | 1.50 | 0.00 | 2.00 | 1.60 | 2.00 | 2.00 |
| S/T coarse Lexus 2 | 30.00 | 71.33 | 71.33 | 34.00 | 62.00 | 70.30 |
| filler | | | | | | |
| S/T fine Lexus 2 filler | 38.00 | 0.00 | 0.00 | 36.00 | 0.00 | 0.00 |
| R812S fumed silica | 2.00 | 2.50 | 2.50 | 2.00 | 4.00 | 2.50 |
| total | 100.00 | 100.00 | 100.00 | 100.00 | 100.00 | 100.00 |

* No HEMA did not have the reactivity for the mixed paste A/B to be cured in short time suitable for RMGI cement.

Example EXA1 Paste A1 was prepared and stored for 35 months at room temperature ambient conditions and was still a stabile paste A with no premature self-polymerization and subsequently was still able to properly react with various pastes B (see below). Example EXA2 Paste A2 was considered a Control Sample, in that it was prepared with no potassium persulfate; EXA2 Paste A2 when combined with EXB1 Paste B1 containing BPBA created a comparative example C.EX-RMGI-1 that exhibited no curing even after 10 minutes, see below.

### “Paste B” Preparations for Resin-Modified Glass Ionomer (RMGI) Dental Compositions

For paste B preparation, AA-MA copolymer was dissolved in water first then BPBA was mixed into the copolymer solution and finally the other ingredients were added for a total amount of 100 grams. Paste B was mixed with a speed mixer at 3000 rpm for 2 minutes and repeated (2 cycles) to form a uniform paste.

TABLE 3
Paste “B” Portion Of RMGI Cement Two-Part System

| | EXB1 | EXB2 | EXB3 | EXB4 |
|---|---|---|---|---|
| RMGI Paste “B” | Paste B1 | Paste B2 | Paste B3 | Paste B4 |
| Components | % | % | % | % |
| DI water | 30.00 | 30.00 | 30.00 | 30.20 |
| AA-MA copolymer | 34.00 | 34.00 | 34.00 | 34.00 |
| DMAPE | 0.00 | 0.20 | 0.20 | 0.15 |
| BPBA | 2.00 | 1.80 | 1.80 | 1.80 |
| 6-micron SiO2 filler | 32.00 | 32.00 | 0.00 | 0.00 |
| Screened YbF3 | 0.00 | 0.00 | 32.00 | 32.00 |
| Silica A300 | 2.00 | 2.00 | 2.00 | 1.85 |
| total | 100.00 | 100.00 | 100.00 | 100.00 |

### Preparation of Resin-Modified Glass Ionomer (RMGI) Cement Examples

Two-part Resin-Modified Glass Ionomer (RMGI) cements were prepared using the above Example Pastes “A” and Pastes “B.” The selected Paste “A” and Paste “B” samples were mixed by hand for 20 seconds in a 6:5-part ratio (A:B respectively; for example 0.42 gram of paste A with 0.35 gram of paste B) on a mixing pad using a dental spatula. The mixed examples were then placed in a 37 °C oven to cure and harden (set). The mixtures were tapped with the spatula to verify they were set, the set time was recorded and is reported below.

TABLE 4
Resin-Modified Glass Ionomer (RMGI) Cement Examples Prepared
By Combining Example Pastes A And Pastes B And
Comparative Example C.EX-RMGI-1

| | | | Set Time |
|---|---|---|---|
| RMGI Example | Paste A | Paste B | min′sec″ |
| C.EX-RMGI-1 | EXA2 Paste A2 | EXB1 Paste B1 | no curing at 10 min |
| | (CONTROL) | | |
| EX-RMGI-1 | EXA3 Paste A3 | EXB1 Paste B1 | 4′45″ |
| EX-RMGI-2 | EXA3 Paste A3 | EXB2 Paste B2 | 4′00 |
| EX-RMGI-3 | EXA3 Paste A3 | EXB3 Paste B3 | 4′00 |
| EX-RMGI-4 | EXA1 Paste A1 | EXB1 Paste B1 | 3′20″ |
| EX-RMGI-5 | EXA1 Paste Al | EXB2 Paste B2 | 3′10″ |
| EX-RMGI-6 | EXA1 Paste A1 | EXB3 Paste B3 | 3′10″ |
| EX-RMGI-7 | EXA4 Paste A4 | EXB1 Paste B1 | 3′30″ |
| EX-RMGI-8 | EXA4 Paste A4 | EXB2 Paste B2 | 3′10″ |
| EX-RMGI-9 | EXA4 Paste A4 | EXB3 Paste B3 | 3′15″ |
| EX-RMGI-10 | EXA4 Paste A4 | EXB4 Paste B4 | 3′10″ |
| EX-RMGI-11 | EXA1 Paste A1 | EXB4 Paste B4 | 3′05″ |

TABLE 5
Resin-Modified Glass Ionomer (RMGI) Cement Examples Prepared By
Combining Example Pastes A And Pastes B,
And Comparative RMGI Examples C.EX-RMGI-2 And C.EX-RMGI-3

| RMGI | | | Set Time |
|---|---|---|---|
| Example | Paste A | Paste B | min'sec″ |
| EX-RMGI-12 | EXA3 Paste A3 | EXB2 Paste B2 | 3'50″ |
| | (Aged 1 month at 45° C.) | | |
| EX-RMGI-13 | EXA3 Paste A3 | EXB3 Paste B3 | 3′50″ |
| | (Aged 1 month at 45° C.) | | |
| C.EX-RMGI-2 | EXA5 Paste A5 | EXB4 Paste B4 | no curing |
| | (No HEMA) | | at 10 min |
| C.EX-RMGI-3 | EXA5 Paste A5 | EXB2 Paste B2 | no curing |
| | (No HEMA) | | at 10 min |

TABLE 6
Resin-Modified Glass Ionomer (RMGI) Cement Examples Prepared
By Combining Various Aged Example Pastes A And Aged Example
Pastes B

| RMGI | | | Set Time |
|---|---|---|---|
| Example | Paste A | Paste B | min'sec″ |
| EX-RMGI-14 | EXA6 Paste A6 | EXB4 Paste B4 | 4′40″ |
| | (Aged 1 month | | |
| | at 45 °C) | | |
| EX-RMGI-15 | EXA6 Paste A6 | EXB4 Paste B4 | 4′20″ |
| | (Aged 1 month | | |
| | at RT) | | |
| EX-RMGI-16 | EXA4 Paste A4 | EXB4 Paste B4 | 2′50″ |
| | (Aged 1 month | (Aged 1 month at 45 °C) | |
| | at 45 °C) | | |
| EX-RMGI-17 | EXA4 Paste A4 | EXB1 Paste B1 | 2′50″ |
| | (Aged 1 month | (Aged 1 month at 45 °C) | |
| | at 45 °C) | | |
| EX-RMGI-18 | EXA4 Paste A4 | EXB2 Paste B2 | 2′50″ |
| | (Aged 1 month | (Aged 1 month at 45 °C) | |
| | at 45 °C) | | |
| EX-RMGI-19 | EXA4 Paste A4 | EXB3 Paste B3 | 2′50″ |
| | (Aged 1 month | (Aged 1 months at 45 °C) | |
| | at 45 °C) | | |
| EX-RMGI-20 | EXA3 Paste A3 | EXB1 Paste B1 | 3′00 |
| | (Aged 2 months | (Aged 1 month at 45 °C) | |
| | at 45 °C) | | |
| EX-RMGI-21 | EXA4 Paste A4 | EXB4 Paste B4 | 2′40″ |
| | (Aged 2 months | (Aged 2 months at 45 °C) | |
| | at 45 °C) | | |
| EX-RMGI-22 | EXA4 Paste A4 | EXB1 Paste B1 | 2′45″ |
| | (Aged 2 months | (Aged 2 months at 45 °C) | |
| | at 45 °C) | | |
| EX-RMGI-23 | EXA4 Paste A4 | EXB2 Paste B2 | 2′40″ |
| | (Aged 2 months | (Aged 2 months at 45 °C) | |
| | at 45 °C) | | |
| EX-RMGI-24 | EXA4 Paste A4 | EXB3 Paste B3 | 2′50″ |
| | (Aged 2 months | (Aged 2 months at 45 °C) | |
| | at 45 °C) | | |
| EX-RMGI-25 | EXA3 Paste A3 | EXB1 Paste B1 | 3′20″ |
| | (Aged 3 months | (Aged 2 months at 45 °C) | |
| | at 45 °C) | | |
| EX-RMGI-26 | EXA4 Paste A4 | EXB4 Paste B4 | 3′30″ |
| | (Aged 5 months | (Aged 5 months at 45 °C) | |
| | at 45 °C) | | |
| EX-RMGI-27 | EXA4 Paste A4 | EXB3 Paste B3 | 3′30″ |
| | (Aged 5 months | (Aged 5 months at 45 °C) | |
| | at 45 °C) | | |
| EX-RMGI-28 | EXA4 Paste A4 | EXB4 Paste B4 | 3′20″ |
| | (Aged 5 months | (Aged 5 months at 45 °C) | |
| | at RT) | | |
| EX-RMGI-29 | EXA4 Paste A4 | EXB3 Paste B3 | 3′20″ |
| | (Aged 5 months | (Aged 5 months at 45 °C) | |
| | at RT) | | |
| EX-RMGI-30 | EXA3 Paste A3 | EXB4 Paste B4 | 3′45″ |
| | (Aged 5 months | (Aged 5 months at 45 °C) | |
| | at 45 °C) | | |
| EX-RMGI-31 | EXA3 Paste A3 | EXB3 Paste B3 | 3′45″ |
| | (Aged 6 months | (Aged 5 months at 45 °C) | |
| | at 45 °C) | | |
| EX-RMGI-32 | EXA1 Paste A1 | EXB3 Paste B3 | 3′00 |
| | | (Aged 5 months at 45 °C) | |

### Light Cure Testing

Individually, Example pastes A and pastes B could not be cured with 3M ELIPAR DeepCure-S LED curing light (wavelength range 430-480 nm) after exposure for 20 seconds due to lack of light cure initiator components in each paste. Once Example pastes A and pastes B are mixed together (0.42 gram of paste A with 0.35 gram of paste B for 20 seconds on mixing pad), thus introducing all light cure initiator components in the RMGI mixture, then the mixture could be light cured to a rubbery or solid state after 20 seconds exposure.

TABLE 7
Resin-Modified Glass Ionomer (RMGI) Cement Examples Prepared
By Combining Example Pastes A And Pastes B And Cured With LED
Blue Light Exposure

| RMGI Example | Paste A | Paste B | Light Curable? |
|---|---|---|---|
| EX-RMGI-33 | EXA4 Paste A4 | EXB4 Paste B4 | yes |
| EX-RMGI-34 | EXA1 Paste A1 | EXB4 Paste B4 | yes |
| EX-RMGI-35 | EXA4 Paste A4 | EXB1 Paste B1 | yes |
| EX-RMGI-36 | EXA4 Paste A4 | EXB2 Paste B2 | yes |
| EX-RMGI-37 | EXA4 Paste A4 | EXB3 Paste B3 | yes |

1. A multi-part hardenable dental composition comprising:
a first part comprising:
a mono- and/or multi-functional component having one or more ethylenically unsaturated group(s),
acid-reactive glass particles; and
an oxidizing agent; and
optionally water, where, if water is present in the first part, it is present in an amount of less than 0.5 wt. % based on a total weight of the first part; and
a second part comprising:
a water miscible polyacid without a polymerizable sidechain group;
a reducing agent comprising a water insoluble organic compound; and
water.

2. The multi-part hardenable dental composition of claim 1, wherein the water insoluble organic compound comprises 1-Benzyl-5-phenyl barbituric acid.

3. The multi-part hardenable dental composition of claim 1, wherein the oxidizing agent comprises at least one of a peroxide, a persulfate, and/or a potassium persulfate.

4. (canceled)

5. (canceled)

6. The multi-part hardenable dental composition of claim 1, wherein the oxidizing agent is present in the first part in an amount of between 0.1 and 4.0 wt. %, based on the total weight the first part.

7. The multi-part hardenable dental composition of claim 1, wherein the acid-reactive glass particles comprise fluoroaluminosilicate (FAS) glass.

8. The multi-part hardenable dental composition of claim 1, wherein the acid-reactive glass particles are present in the first part in an amount of between 3 and 60 wt. %, based on the total weight the first part.

9. (canceled)

10. The multi-part hardenable dental composition of claim 1, wherein the polyacid comprises homo- or copolymers of acrylic acid, itaconic acid or maleic acid.

11. The multi-part hardenable dental composition of claim 1, wherein the polyacid is of the formula:
B(X)ₘ(Y)ₙ
wherein B is an organic backbone, each X independently is an ionic group which can undergo a setting reaction in the presence of water and the acid-reactive glass particles, each Y independently is a non-ionically polymerizable group, m is at least 2, and n is at least 1.

12. The multi-part hardenable dental composition of claim 11, X is —COOH and Y is a copolymer group that does not include an ethylenically unsaturated group.

13. The multi-part hardenable dental composition of claim 1, wherein the water miscible polyacid is present in the second part in an amount of between 5 and 35 wt. %, based on the total weight the second part.

14. The multi-part hardenable dental composition of claim 1, wherein the reducing agent is present in the second part in an amount of between 0.25 and 5.0 wt. %, based on the total weight the second part.

15. The multi-part hardenable dental composition of claim 1, wherein water is present in the second part in an amount of between 1 and 70 wt. %, based on the total weight the second part.

16. The multi-part hardenable dental composition of claim 1, wherein the first part, the second part, or both of the first and second parts comprise non-reactive filler in an amount of 1 to 40 weight percent, based upon the total weight of the part which includes the non-reactive filler.

17. The multi-part hardenable dental composition of claim 1, wherein the second part further comprise non-reactive filler.

18. The multi-part hardenable dental composition of claim 17, wherein the non-reactive filler comprises inorganic material, crosslinked organic material, and a combination thereof.

19. The multi-part hardenable dental composition of claim 17, wherein the second part includes the non-reactive filler in an amount of 30 to 40 weight percent, based upon the total weight of the second part.

20. The multi-part hardenable dental composition of claim 17, wherein the non-reactive filler comprises fumed silica, zirconia-silica, quartz, nonpyrogenic silica, or a combination thereof.

21. The multi-part hardenable dental composition of claim 1, wherein the mono- and/or multi-functional components of the first part are liquid at room temperature and water miscible.

22. The multi-part hardenable dental composition of claim 1, wherein the first part comprises both mono- and multi-functional components having ethylenically unsaturated group(s).

23. The multi-part hardenable dental composition of claim 22, wherein the mono- and multi-functional components having ethylenically unsaturated group(s) of the first part are present in their first part in an amount of between 3 and 40 wt. %, based on the total weight the first part.

24. The multi-part hardenable dental composition of claim 1, wherein upon mixing, the multi-part hardenable dental composition has a set time of less than 10 minutes.

25. The multi-part hardenable dental composition of claim 1, wherein the multi-part hardenable dental composition is selected from the group consisting of a liner material, a luting material, a restorative material, an endodontic material, and a sealing material.

26. The multi-part hardenable dental composition of claim 1, wherein either or both of the first and second parts are a paste.

27. A method of using the multi-part hardenable dental composition of claim 1, the method comprising:
adhering a dental article to a tooth or bone using the multi-part hardenable dental composition of claim 1; or
at least partially filling a cavity of a tooth with the multi-part hardenable dental composition of claim 1.
